(19) United States
(12) Patent Application Publication
(10) Pub. No.: US 2019/0093140 A1
(43) Pub. Date: Mar. 28, 2019

(54) ENZYMATIC SYSTEMS AND METHODS FOR SYNTHESIZING NICOTINAMIDE MONONUCLEOTIDE AND NICOTINIC ACID MONONUCLEOTIDE

(71) Applicants: NEWSOUTH INNOVATIONS PTY LIMITED, Sydney, NSW (AU); METRO BIOTECH, LLC, Birmingham, MI (US)

(72) Inventors: Lindsay Wu, Waterloo (AU); David A. Sinclair, Chestnut Hill, MA (US); Kyle Meetze, Lexington, MA (US)

(21) Appl. No.: 15/580,972

(22) PCT Filed: Jun. 8, 2016
(86) PCT No.: PCT/IB2016/000874

§ 371 Date: Dec. 8, 2017

Related U.S. Application Data
(60) Provisional application No. 62/174,412, filed on Jun. 11, 2015.

(51) Int. Cl. C12P 19/30; C12P 19/36; C12N 11/18; C12N 9/12; C12N 9/10; C12N 11/08

(52) U.S. Cl. CPC C12P 19/30; C12P 19/36; C12Y 204/02012; C12Y 204/02011; C12N 11/08; C12Y 207/06001; C12N 9/1235; C12N 9/1077; C12N 11/18

(57) ABSTRACT

Enzyme-based systems and methods for synthesizing the NAD precursors NMN and NaMN are disclosed. Such methods and systems utilize a mutated form of phosphoribosylpyrophosphate synthetase (PRS) that is superactive and/or other enzyme or enzyme combinations that are immobilized onto a solid surface. The methods and systems substantially increase the efficiency and yield of NAD precursor synthesis.

### CROSS-REFERENCE TO RELATED APPLICATIONS

This application claims priority to U.S. Provisional Patent Application No. 62/174,412, filed Jun. 11, 2015, which is incorporated herein by reference as if set forth in its entirety.

### STATEMENT REGARDING FEDERALLY SPONSORED RESEARCH OR DEVELOPMENT

Not Applicable

### BACKGROUND

Nicotinamide Adenine Dinucleotide (NAD⁺) is an essential metabolic cofactor. Recent research has indicated that NAD⁺ levels decline with age and in certain mammalian disease states, and that therapeutically increasing NAD⁺ levels has health benefits. However, NAD⁺ is an intracellular metabolite, and does not readily lend itself to external supplementation. It has been suggested that utilizing precursors to the natural synthesis of NAD⁺ may be an effective way to increase NAD⁺.

Two exemplary precursors that could be administered to increase NAD⁺ are nicotinamide monomucleotide (NMN), which is directly synthesized into NAD⁺, and nicotinamide riboside (NR), which is recycled from the utilization of NAD⁺, into NMN. There are no known dietary or environmental sources of NMN or NR. Accordingly, in order to use these precursors as drugs or supplements, they must be manufactured.

U.S. Pat. No. 8,106,184, issued to Sauve et. al., describes methods for the efficient manufacture of NR through synthetic chemistry. No such method exists for the production of NMN. U.S. Pat. No. 4,411,995, issued to Whitesides and Walt, describes an enzymatic process for producing NMN, but such a method, while efficient in its yield, requires carefully controlled conditions and the addition of costly enzymes.

Therefore a need exists in the art for an improved method to manufacture NAD precursors at high yield, high purity, and lower cost. Such methods are described herein.

### BRIEF SUMMARY

This disclosure relates to new methods and systems for the enzymatic synthesis of the NAD precursors, such as nicotinamide mononucleotide (NMN) and nicotinic acid mononucleotide (NaMN). Various enzyme-based methods for the production of NMN have been described by, for example, Whitesides (1985), Berghauser (1981), Dietrich (1966), and Preiss (1957). Here we disclose enzyme-based systems and methods for producing NMN or NaMN utilizing one or more improvements. Specifically, the disclosed systems and methods utilize (1) a mutated form of phosphoribosylpyrophosphate synthetase (PRS) that is rendered insensitive to its own reaction product, thus increasing its activity; and/or (2) one or more other enzymes or enzyme combinations (optionally including the mutated form of PRS) that are bound to a solid surface. The one or more enzymes used may be produced by recombinant means in one or more cells, including, without limitation, in yeast, bacteria, baculovirus, or mammalian cell lines. Alternatively, the one or more enzymes used may be produced in cell-containing or cell-free in vitro translation systems, such as in reticulocyte lysate.

Accordingly, in a first aspect, the disclosure encompasses a system for synthesizing an nicotinamide adenine dinucleotide (NAD) precursor. The system includes a superactive phosphoribosylpyrophosphate synthetase (PRS) mutant, wherein the PRS mutant is less sensitive to the product of the reaction that it catalyzes than a wild type PRS.

In some embodiments, the superactive PRS mutant includes a polypeptide that differs from wild type PRS by one or more amino acid substitutions. In some such embodiments, the one or more amino acid substitutions are Asp51His of human PRS, Asn113Ser of human PRS, Leu128Ile of human PRPP, Asp182His of human PRS, Ala189Val of human PRS, His192Gln of human PRS, any of the equivalent substitutions in a non-human PRS, or any combination of these.

In some embodiments, the superactive PRS mutant includes one or more affinity tags. In some such embodiments, the affinity tag is a 6xHis tag or a glutathione S-transferase (GST) tag.

In some embodiments, the superactive PRS mutant is recombinantly produced, isolated, or purified from cells.

In some embodiments, the superactive PRS mutant is immobilized onto a surface. In some such embodiments, the superactive PRS mutant is immobilized onto the surface by adsorption, affinity binding, ionic bonding, or covalent bonding. In some embodiments, the surface is the surface of a bead or comprises a resin.

In some embodiments, the system further includes nicotinamide phosphoribosyltransferase (NAMPT) or nicotinate phosphoribosyltransferase (NAPRT). In some such embodiments, the NAMPT or NAPRT includes one or more affinity tags. In some such embodiments, the affinity tag is a 6xHis tag or a glutathione S-transferase (GST) tag.

In some embodiments, the NAMPT or NAPRT is recombinantly produced, isolated, or purified from cells.

In some embodiments, the NAMPT or NAPRT is immobilized onto a surface. In some such embodiments, the NAMPT or NAPRT is immobilized onto the surface by adsorption, affinity binding, ionic bonding, or covalent bonding. In some embodiments, the surface is the surface of a bead or comprises a resin.

In some embodiments where the NAMPT or NAPRT is immobilized onto a surface, the PRS mutant is also immobilized onto a surface. In some such embodiments, the PRS mutant and the NAMPT or NAPRT are immobilized onto different surfaces. In other such embodiments, the PRS mutant and the NAMPT or NAPRT are immobilized onto the same surface.

In some embodiments, the system further includes adenosine triphosphate (ATP).

In some embodiments, the system further includes ribose-5-phosphate.

In some embodiments, the system further includes nicotinamide or nicotinic acid.

In some embodiments, the system further includes phosphoribosyl pyrophosphate (PRPP).

In some embodiments, the system further includes nicotinamide mononucleotide (NMN) or nicotinic acid mononucleotide (NaMN).

In a second aspect, this disclosure encompasses a method for synthesizing an nicotinamide adenine dinucleotide (NAD) precursor. The method includes the step of contacting ribose-5-phosphate with a superactive phosphoribosylpyrophosphate synthetase (PRS) mutant in the presence of adenosine triphosphate (ATP), wherein the PRS mutant is less sensitive to the product of the reaction that it catalyzes than a wild type PRS, and whereby phosphoribosyl pyrophosphate (PRPP) is produced.

In some embodiments, the superactive PRS mutant comprises a polypeptide that differs from wild type PRS by one or more amino acid substitutions. In some such embodiments, the one or more amino acid substitutions can be Asp51His of human PRS, Asn113Ser of human PRS, Leu128Ile of human PRPP, Asp182His of human PRS, Ala189Val of human PRS, His192Gln of human PRS, any of the equivalent substitutions in a non-human PRS, and any combination of these.

In some embodiments, the superactive PRS mutant includes one or more affinity tags. In some such embodiments, the affinity tag is a 6xHis tag or a glutathione S-transferase (GST) tag.

In some embodiments, the superactive PRS mutant is recombinantly produced, isolated, or purified from cells.

In some embodiments, the superactive PRS mutant is immobilized onto a surface. In some such embodiments, the superactive PRS mutant is immobilized onto the surface by adsorption, affinity binding, ionic bonding, or covalent bonding.

In some embodiments, the surface is the surface of a bead or comprises a resin.

In some embodiments, the method further includes the steps of (a)contacting the resulting PRPP with nicotinamide phosphoribosyltransferase (NAMPT) in the presence of nicotinamide, whereby nicotinamide mononucleotide (NMN) is produced; or (b) contacting the resulting PRPP with nicotinate phosphoribosyltransferase (NAPRT) in the presence of nicotinic acid, whereby nicotinic acid mononucleotide (NaMN) is produced.

In some embodiments, the NAMPT or NAPRT include one or more affinity tags. In some such embodiments, the affinity tag is a 6xHis tag or a glutathione S-transferase (GST) tag.

In some embodiments, the NAMPT or NAPRT is recombinantly produced, isolated, or purified from cells.

In some embodiments, the NAMPT or NAPRT is immobilized onto a surface. In some such embodiments, the NAMPT or NAPRT is immobilized onto the surface by adsorption, affinity binding, ionic bonding, or covalent bonding. In some such embodiments, the surface is the surface of a bead or comprises a resin.

In some embodiments, the PRS mutant is also immobilized onto a surface. In some such embodiments, the PRS mutant and the NAMPT or NAPRT are immobilized onto different surfaces. In other such embodiments, the PRS mutant and the NAMPT or NAPRT are immobilized onto the same surface.

In some embodiments, the method further includes the step of purifying or concentrating the NMN or NaMN produced.

In a third aspect, this disclosure encompasses a system for synthesizing nicotinamide mononucleotide (NMN). The system includes nicotinamide riboside kinase (NRK) enzyme immobilized onto a surface.

In some embodiments the NRK includes one or more affinity tags. In some such embodiments, the affinity tag is a 6xHis tag or a glutathione S-transferase (GST) tag.

In some embodiments, the NRK is recombinantly produced, isolated, or purified from a cell.

In some embodiments, the NRK is immobilized onto the surface by adsorption, affinity binding, ionic bonding, or covalent bonding.

In some embodiments, the surface is the surface of a bead or comprises a resin.

In some embodiments, the NRK is purified from cells or produced through recombinant means.

In some embodiments, the system further includes adenosine triphosphate (ATP).

In some embodiments, the system further includes nicotinamide riboside.

In some embodiments, the system further includes nicotinamide mononucleotide (NMN).

In a fourth aspect, this disclosure encompasses a method for synthesizing nicotinamide mononucleotide (NMN). The method includes the steps of contacting nicotinamide riboside kinase (NRK) immobilized onto a surface with nicotinamide riboside in the presence of adenosine triphosphate (ATP), whereby NMN is produced.

In some embodiments, the NRK includes one or more affinity tags. In some such embodiments, the affinity tag is a 6xHis tag or a glutathione S-transferase (GST) tag.

In some embodiments, the NRK is recombinantly produced, isolated, or purified from a cell.

In some embodiments, the NRK is immobilized onto the surface by adsorption, affinity binding, ionic bonding, or covalent bonding. In some embodiments, the surface is the surface of a bead or comprises a resin.

Some embodiments further include the step of purifying or concentrating the NMN produced.

In a fifth aspect, this disclosure encompasses a system for synthesizing nicotinamide mononucleotide (NMN). The system includes the following enzymes immobilized onto a surface: (a) a superactive phosphoribosylpyrophosphate synthetase (PRS) mutant, wherein the PRS mutant is less sensitive to the product of the reaction that it catalyzes than a wild type PRS; (b) hexokinase; (c) glucose-6phosphate dehydrogenase; (d) gluconolactonase; (e) 6-phospho gluconate dehydrogenase; (f) ribulose-5-phosphate isomerase; and (g) nicotinamide phosphoribosyl transferase.

In some embodiments, one or more of the immobilized enzymes include one or more affinity tags. In some such embodiments, the affinity tag is a 6xHis tag or a glutathione S-transferase (GST) tag.

In some embodiments, one or more of the immobilized enzymes is recombinantly produced, isolated, or purified from a cell.

In some embodiments, the NRK is immobilized onto the surface by adsorption, affinity binding, ionic bonding, or covalent bonding.

In some embodiments, the superactive PRS mutant includes a polypeptide that differs from wild type PRS by one or more amino acid substitutions. In some such embodiments, the one or more amino acid substitutions can be Asp51His of human PRS, Asn113Ser of human PRS, Leu128Ile of human PRPP, Asp182His of human PRS, Ala189Val of human PRS, His192Gln of human PRS, any of the equivalent substitutions in a non-human PRS, or any combination of these.

In some embodiments, the surface is the surface of a bead or comprises a resin.

In some embodiments, each enzyme is immobilized onto a different surface. In other embodiments, each enzyme is immobilized onto a different surface. In yet other embodiments, the six immobilized enzymes are immobilized to between two and five different surfaces.

In some embodiments, the system may also include one or more of glucose, nicotinamide, adenosine triphosphate (ATP), nicotinamide adenine dinucleotide phosphate (NADP⁺), an oxidizing agent, or mixtures thereof.

In a sixth aspect, this disclosure encompasses a method for synthesizing nicotinamide mononucleotide (NMN). The method includes the step of contacting the system described in any of the previous eight paragraphs with nicotinamide in the presence of glucose, adenosine triphosphate (ATP), Nicotinamide adenine dinucleotide phosphate (NADP⁺), and an oxidizing agent, whereby NMN is produced.

In some embodiments, the method further includes the step of purifying or concentrating the NMN produced.

### DETAILED DESCRIPTION

### I. Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The terms “optional” or “optionally” as used herein means that a subsequently described event or circumstance may but need not occur, and that the description includes instances where the event or circumstance occurs and instances in which it does not. For example, “optional bond” means that the bond may or may not be present, and that the description includes single, double, or triple bonds.

The term “purified,” as described herein, refers to the purity of a given compound. For example, a compound is “purified” when the given compound is a major component of the composition, i.e., at least 50% w/w pure. Thus, “purified” embraces at least 50% w/w purity, at least 60% w/w purity, at least 70% purity, at least 80% purity, at least 85% purity, at least 90% purity, at least 92% purity, at least 94% purity, at least 96% purity, at least 97% purity, at least 98% purity, at least 99% purity, at least 99.5% purity, and at least 99.9% purity, wherein “substantially pure” embraces at least 97% purity, at least 98% purity, at least 99% purity, at least 99.5% purity, and at least 99.9% purity.

“Nicotinamide,” which corresponds to the following structure,

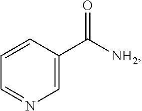

is one of the two principal forms of the B-complex vitamin niacin. The other principal form of niacin is nicotinic acid; nicotinamide, rather than nicotinic acid, however, is the major substrate for nicotinamide adenine dinucleotide (NAD) biosynthesis in mammals, as discussed in detail herein. Nicotinamide, in addition to being known as niacinamide, is also known as 3-pyridinecarboxamide, pyridine-3-carboxamide, nicotinic acid amide, vitamin B3, and vitamin PP. Nicotinamide has a molecular formula of C₆H₆N₂O and its molecular weight is 122.13 Daltons. Nicotinamide is commercially available from a variety of sources.

“Nicotinamide Adenine Dinucleotide” (NAD⁺), which corresponds to the following structure,

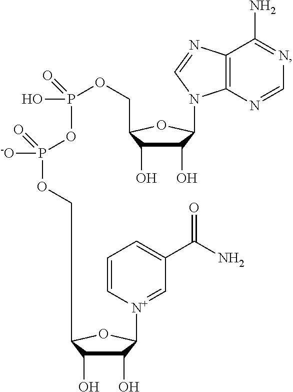

is produced from the conversion of nicotinamide to NMN, which is catalyzed by Nampt, and the subsequent conversion of NMN to NAD, which is catalyzed by Nmnat. Nicotinamide adenine dinucleotide (NAD) has a molecular formula of C₂₁H₂₇N₇O₁₄P₂ and a molecular weight of 663.43. Nicotinamide adenine dinucleotide (NAD) is commercially available from such sources as Sigma-Aldrich (St. Louis, Mo.).

“Nicotinamide Mononucleotide” (NMN), which corresponds to the following structure,

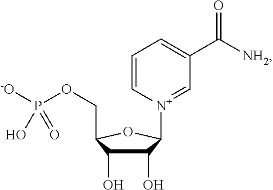

is produced from nicotinamide in the NAD biosynthesis pathway, a reaction that is catalyzed by Nampt. NMN is further converted to NAD in the NAD biosynthesis pathway, a reaction that is catalyzed by Nmnat. Nicotinamide mononucleotide (NMN) has a molecular formula of C₁₁H₁₅N₂O₈P and a molecular weight of 334.22. Nicotinamide mononucleotide (NMN) is commercially available from such sources as Sigma-Aldrich (St. Louis, Mo.).

“Nicotinamide Riboside” (NR), which corresponds to the following structure,

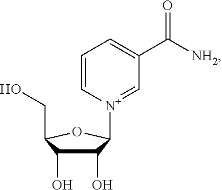

is characterized and a synthesized as described in, for instance, U.S. Pat. No. 8,106,184.

“Nicotinic Acid Mononucleotide” (NaMN) corresponds to the following structure:

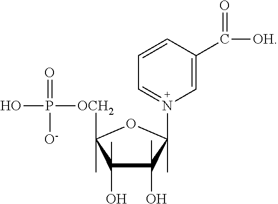

“Nicotinic Acid Riboside” (NaR) corresponds to the following structure:

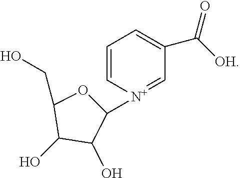 [SMILES: O=C(O)c1ccc[n+](C2OC(CO)C(O)C2O)c1]

### II. Description of Selected Exemplary Embodiments

The following exemplary methods and systems are offered for illustrative purposes only, and are not intended to limit the scope of the present invention in any way. Indeed, various modifications of the invention in addition to those shown and described herein will become apparent to those skilled in the art from the foregoing description and the following exemplary systems and methods.

### Exemplary Method 1

The human enzyme phosphoribosoylpyrophosphate synthetase (PRS) is mutated to increase its activity through rendering it insensitive to the product of its own reaction, phosphoribosyl pyrophosphate (PRPP). Mutations may include, without limitation, Asp51His, Asn113Ser, Leu128Ile, Asp182His, Ala189Val and His192Gln. These mutations are defined relative to the known sequence of human PRS. However, PRS from other species may be used in the disclosed systems and methods, with equivalent mutations in non-human homologs also resulting in the required superactivity.

Enzymes may optionally be tagged with affinity tags, such as 6xHis tag or GST tag. Recombinant or purified enzyme mutants as described above may be immobilized on, for example, beads or resin (e.g., agarose beads, sepharose beads) through adsorption, affinity binding (e.g. 6xHis tagged proteins to Ni²⁺ or Co²⁺ beads), ionic binding or covalent bonds. Such methods are well-known in the art.

Ribose-5-phosphate in the presence of ATP may be passed through beads or resin with immobilized, mutated PRS to yield PRPP. The enzyme nicotinamide phosphoribosyltransferase (NAMPT) may be tagged and immobilized to beads or resin, as described above and known in the art. The product of the previous reaction can be combined with nicotinamide and passed through such a resin to yield nicotinamide mononucleotide (NMN).

In an alternative method, resin or beads carrying recombinant or isolated NAMPT are placed in a bottom layer of a column, and resin or beads carrying recombinant or isolated PRS mutants are placed in an upper layer of a column. A single mixture containing nicotinamide, ribose-5-phosphate and ATP is then passed through the column to yield NMN as a final product.

In another alternative method, resin or beads carrying immobilized PRS mutant enzyme and NAMPT enzyme are mixed into a single column, and a mixture containing nicotinamide, ribose-5-phosphate and ATP is then passed through the column. This latter embodiment will have the advantage of consuming PRPP to further reduce inhibition of PRS enzyme.

In yet another alternative method, NAMPT is replaced by nicotinate phosphoribosyltransferase and nicotinamide is replaced by nicotinic acid, to yield nicotinic acid mononucleotide (NaMN).

### Exemplary Method 2

The enzyme nicotinamide riboside kinase (NRK) may be purified from cells or produced through recombinant means, and then immobilized on a solid support (e.g. resin, beads). Nicotinamide riboside and ATP are then passed over this solid support to yield nicotinamide mononucleotide (NMN).

### Exemplary Method 3

Glucose, nicotinamide, ATP, NADP⁺and an oxidizing agent are passed over a solid support (e.g. resin, beads) which contain the isolated or recombinant enzymes hexokinase, glucose-6-phosphate dehydrogenase, gluconolactonase, 6-phospho gluconate dehydrogenase, ribulose-5-phosphate isomerase, mutant versions of phosphoriboylpyrophosphatase synthetase, and nicotinamide phosphoribosyl transferase. These enzymes may be immobilized to separate solid supports, and placed in layers in the order listed above from top to bottom. Alternatively, all enzymes may be mixed and immobilized to the same solid support. Glucose and nicotinamide will be converted by these enzymes into NMN, which will consume ATP and require the conversion of NADP⁺ into NADPH. NADPH will be immediately regenerated back into NADP⁺ through the addition of an oxidizing agent.

The midpoint potential of the NADP⁺/NADPH redox pair is −0.324 volts, meaning that NADPH is comparatively easy to oxidize. Thus, preferred oxidizing agents may include any of a number of very mild oxidizing agents known in the art to be capable of oxidizing NADPH into NADP⁺.

The enzymes used in the disclosed systems and methods in all of the above-disclosed exemplary methods may be produced through recombinant means in microbes, such as in yeast, bacteria, baculovirus, or in eukaryotic cells, such as in mammalian cell lines. Methods of producing recombinant enzymes using such host cells are well-known in the art. Alternatively, the enzymes may be produced through in vitro translation methods. A variety of cell-free translation methods are known in the art. A non-limiting example is the use of reticulocyte lysate to facilitate enzyme production.

Constructs for recombinant expression may be subjected to codon optimization from the parent cDNA to increase protein translation. Again, such techniques are well-known in the art. Although the enzymes described in this disclosure are the human forms, the human form of the enzymes used may be substituted with the orthologous enzymes from other species, depending on the efficiency of their activity.

Other embodiments and uses will be apparent to those skilled in the art from consideration from the specification and practice of the invention disclosed herein. It is understood that the invention is not confined to the specific reagents, formulations, reaction conditions, etc., herein illustrated and described, but embraces such modified forms thereof as come within the scope of the following claims.

All references cited herein for any reason, including all journal citations and U.S./foreign patents and patent applications, are specifically and entirely incorporated by reference herein.

1. A system for synthesizing a nicotinamide adenine dinucleotide (NAD) precursor, the system comprising a superactive phosphoribosylpyrophosphate synthetase (PRS) mutant, wherein the PRS mutant is less sensitive to the product of the reaction that it catalyzes than a wild type PRS.

2. The system of claim 1, wherein the superactive PRS mutant comprises a polypeptide that differs from wild type PRS by one or more amino acid substitutions.

3. The system of claim 1 or claim 2, wherein the one or more amino acid substitutions are selected from the group consisting of Asp51His of human PRS, Asn113Ser of human PRS, Leu128Ile of human PRPP, Asp182His of human PRS, Ala189Val of human PRS, His192Gln of human PRS, any of the equivalent substitutions in a non-human PRS, and any combination thereof.

4. The system of any of claims 1-3, wherein the superactive PRS mutant comprises one or more affinity tags.

5. The system of claim 4, wherein the affinity tag is a 6xHis tag or a glutathione S-transferase (GST) tag.

6. The system of any of claims 1-5, wherein the superactive PRS mutant is recombinantly produced, isolated, or purified.

7. The system of any of claims 1-6, wherein the superactive PRS mutant is immobilized onto a surface.

8. The system of claim 7, wherein the superactive PRS mutant is immobilized onto the surface by adsorption, affinity binding, ionic bonding, or covalent bonding.

9. The system of claim 7 or claim 8, wherein the surface is the surface of a bead or comprises a resin.

10. The system of any of claims 1-9, further comprising nicotinamide phosphoribosyltransferase (NAMPT) or nicotinate phosphoribosyltransferase (NAPRT).

11. The system of claim 10, wherein the NAMPT or NAPRT comprises one or more affinity tags.

12. The system of claim 11, wherein the affinity tag is a 6xHis tag or a glutathione S-transferase (GST) tag.

13. The system of any of claims 10-13, wherein the NAMPT or NAPRT is recombinantly produced, isolated, or purified.

14. The system of any of claims 10-13, wherein the NAMPT or NAPRT is immobilized onto a surface.

15. The system of claim 14, wherein the NAMPT or NAPRT is immobilized onto the surface by adsorption, affinity binding, ionic bonding, or covalent bonding.

16. The system of claim 14 or claim 15, wherein the surface is the surface of a bead or comprises a resin.

17. The system of any of claims 14-16, wherein, the PRS mutant is also immobilized onto a surface.

18. The system of claim 17, wherein the PRS mutant and the NAMPT or NAPRT are immobilized onto different surfaces.

19. The system of claim 17, wherein the PRS mutant and the NAMPT or NAPRT are immobilized onto the same surface.

20. The system of any of claim 1-19, further comprising adenosine triphosphate (ATP).

21. The system of any of claims 1-20, further comprising ribose-5-phosphate.

22. The system of any of claims 1-21, further comprising nicotinamide or nicotinic acid.

23. The system of any of claims 1-22, further comprising phosphoribosyl pyrophosphate (PRPP).

24. The system of any of claims 1-23, further comprising nicotinamide mononucleotide (NMN) or nicotinic acid mononucleotide (NaMN).

25. A method for synthesizing an nicotinamide adenine dinucleotide (NAD) precursor comprising contacting ribose-5-phosphate with a superactive phosphoribosylpyrophosphate synthetase (PRS) mutant in the presence of adenosine triphosphate (ATP), wherein the PRS mutant is less sensitive to the product of the reaction that it catalyzes than a wild type PRS, and whereby phosphoribosyl pyrophosphate (PRPP) is produced.

26. The method of claim 25, wherein the superactive PRS mutant comprises a polypeptide that differs from wild type PRS by one or more amino acid substitutions.

27. The method of claim 25 or claim 26, wherein the one or more amino acid substitutions are selected from the group consisting of Asp51His of human PRS, Asn113Ser of human PRS, Leu128Ile of human PRPP, Asp182His of human PRS, Ala189Val of human PRS, His192Gln of human PRS, any of the equivalent substitutions in a non-human PRS, and any combination thereof.

28. The method of any of claims 25-27, wherein the superactive PRS mutant comprises one or more affinity tags.

29. The method of claim 28, wherein the affinity tag is a 6xHis tag or a glutathione S-transferase (GST) tag.

30. The method of any of claims 25-29, wherein the superactive PRS mutant is recombinantly produced, isolated, or purified.

31. The method of any of claims 25-30, wherein the superactive PRS mutant is immobilized onto a surface.

32. The method of claim 31, wherein the superactive PRS mutant is immobilized onto the surface by adsorption, affinity binding, ionic bonding, or covalent bonding.

33. The method of claim 31 or claim 32, wherein the surface is the surface of a bead or comprises a resin.

34. The method of any of claims 25-33, further comprising:
(a) contacting the resulting PRPP with nicotinamide phosphoribosyltransferase (NAMPT) in the presence of nicotinamide, whereby nicotinamide monocleotide (NMN) is produced; or
(b) contacting the resulting PRPP with nicotinate phosphoribosyltransferase (NAPRT) in the presence of nicotinic acid, whereby nicotinic acid mononucleotide (NaMN) is produced.

35. The method of claim 34, wherein the NAMPT or NAPRT comprises one or more affinity tags.

36. The method of claim 35, wherein the affinity tag is a 6xHis tag or a glutathione S-transferase (GST) tag.

37. The method of any of claims 34-36, wherein the NAMPT or NAPRT is recombinantly produced, isolated, or purified.

38. The method of any of claims 34-37, wherein the NAMPT or NAPRT is immobilized onto a surface.

39. The method of claim 38, wherein the NAMPT or NAPRT is immobilized onto the surface by adsorption, affinity binding, ionic bonding, or covalent bonding.

40. The method of claim 38 or claim 39, wherein the surface is the surface of a bead or comprises a resin.

41. The method of any of claims 38-40, wherein, the PRS mutant is also immobilized onto a surface.

42. The method of claim 41, wherein the PRS mutant and the NAMPT or NAPRT are immobilized onto different surfaces.

43. The method of claim 17, wherein the PRS mutant and the NAMPT or NAPRT are immobilized onto the same surface.

44. The method of any of claims 34-43, further comprising purifying or concentrating the NMN or NaMN produced.

45. A system for synthesizing nicotinamide mononucleotide (NMN), the system comprising nicotinamide riboside kinase (NRK) immobilized onto a surface.

46. The system of claim 45, wherein the NRK comprises one or more affinity tags.

47. The system of claim 46, wherein the affinity tag is a 6xHis tag or a glutathione S-transferase (GST) tag.

48. The system of any of claims 45-47, wherein the NRK is recombinantly produced, isolated, or purified.

49. The system of any of claims 45-48, wherein the NRK is immobilized onto the surface by adsorption, affinity binding, ionic bonding, or covalent bonding.

50. The system of any of claims 45-49, wherein the surface is the surface of a bead or comprises a resin.

51. the system of any of claims 45-50, wherein the NRK is purified from cells or produced through recombinant means.

52. The system of any of claims 45-51, further comprising adenosine triphosphate (ATP).

53. The system of any of claims 45-52, further comprising nicotinamide riboside.

54. The system of any of claims 45-53, further comprising nicotinamide mononucleotide (NMN).

55. A method for synthesizing nicotinamide mononucleotide (NMN), the method comprising contacting nicotinamide riboside kinase (NRK) immobilized onto a surface with nicotinamide riboside in the presence of adenosine triphosphate (ATP), whereby NMN is produced.

56. The method of claim 55, wherein the NRK comprises one or more affinity tags.

57. The method of claim 56, wherein the affinity tag is a 6xHis tag or a glutathione S-transferase (GST) tag.

58. The method of any of claims 55-57, wherein the NRK is recombinantly produced, isolated, or purified.

59. The method of any of claims 55-58, wherein the NRK is immobilized onto the surface by adsorption, affinity binding, ionic bonding, or covalent bonding.

60. The method of any of claims 55-59, wherein the surface is the surface of a bead or comprises a resin.

61. The method of any of claims 55-60, further comprising purifying or concentrating the NMN produced.

62. A system for synthesizing nicotinamide mononucleotide (NMN), the system comprising the following enzymes immobilized onto a surface:
(a) a superactive phosphoribosylpyrophosphate synthetase (PRS) mutant, wherein the PRS mutant is less sensitive to the product of the reaction that it catalyzes than a wild type PRS;
(b) hexokinase;
(c) glucose-6phosphate dehydrogenase;
(d) gluconolactonase;
(e) 6-phospho gluconate dehydrogenase;
(f) ribulose-5-phosphate isomerase; and
(g) nicotinamide phosphoribosyl transferase.

63. The system of claim 62, wherein one or more of the immobilized enzymes comprises one or more affinity tags.

64. The system of claim 63, wherein the affinity tag is a 6xHis tag or a glutathione S-transferase (GST) tag.

65. The system of any of claims 62-64, wherein one or more of the immobilized enzymes is recombinantly produced, isolated, or purified from a cell.

66. The system of any of claims 62-65, wherein the NRK is immobilized onto the surface by adsorption, affinity binding, ionic bonding, or covalent bonding.

67. The system of any of claims 62-66, wherein the superactive PRS mutant comprises a polypeptide that differs from wild type PRS by one or more amino acid substitutions.

68. The system of claim 67, wherein the one or more amino acid substitutions are selected from the group consisting of Asp51His of human PRS, Asn113Ser of human PRS, Leu128Ile of human PRPP, Asp182His of human PRS, Ala189Val of human PRS, His192Gln of human PRS, any of the equivalent substitutions in a non-human PRS, and any combination thereof.

69. The system of any of claims 62-68, wherein the surface is the surface of a bead or comprises a resin.

70. The system of any of claims 62-69, wherein each enzyme is immobilized onto a different surface.

71. The system of any of claims 62-69, wherein each enzyme is immobilized onto a different surface.

72. The system of any of claims 62-69, wherein the six immobilized enzymes are immobilized to between two and five different surfaces.

73. The system of any of claims 62-72, further comprising one or more of the group consisting of glucose, nicotinamide, adenosine triphosphate (ATP), Nicotinamide adenine dinucleotide phosphate (NADP⁺), an oxidizing agent, and mixtures thereof.

74. A method for synthesizing nicotinamide mononucleotide (NMN), the method comprising contacting the system of any of claims 62-72 with nicotinamide in the presence of glucose, adenosine triphosphate (ATP), Nicotinamide adenine dinucleotide phosphate (NADP⁺), and an oxidizing agent;
whereby NMN is produced.

75. The method of claim 74, further comprising purifying or concentrating the NMN produced.
